(19) United States
(12) Patent Application Publication
(10) Pub. No.: US 2008/0058550 A1
(43) Pub. Date: Mar. 6, 2008

(54) Chemical process

(75) Inventors: Ian Patel, Bristol (GB); Neil Smith, Bristol (GB); Simon Nicholas George Tyler, Bristol (GB)

Correspondence Address: FISH & RICHARDSON P.C., P.O BOX 1022, MINNEAPOLIS, MN, 55440-1022, US

(73) Assignee: ASTRAZENECA AB

(21) Appl. No.: 11/897,688

(22) Filed: Aug. 31, 2007

(30) Foreign Application Priority Data
Sep. 2, 2006 (GB) 0617366.0

(51) Int. Cl. C07C 291/00

(52) U.S. Cl. 564/298

(57) ABSTRACT

A process for preparing a salt of compound of formula (I) 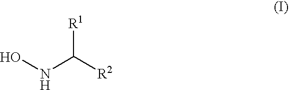
wherein R¹ and R² are independently selected from an organic group other than hydrogen, said process comprising reacting a compound of formula (II) 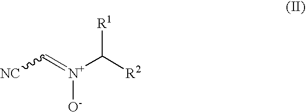 with water and an organic acid, in the absence of hydroxylamine. The reaction is useful in preparing a range of chemical intermediates, in particular chiral compounds.

The present invention relates to a process for the synthesis of a chiral hydroxylamine, useful as an intermediate in the production of a range of chemicals, and in particular pharmaceutical compounds.

Hydroxylamine compounds have a wide range of applications in particular as intermediates in the production of pharmaceutical compounds. Such compounds are frequently required to be enantiomerically pure or at least to contain a preponderance of a single enantiomer.

Resolution of racemic or other mixtures is often time consuming and wasteful, and is not generally suitable for large scale production methods. Therefore stereoselective reaction methods are generally sought.

One such method, which can maintain chiral integrity if it utilizes resolved starting materials, is described by H. Tokuyama et al., Synthesis 2000, No. 9, 1299-1304 and H. Tokuyama et al., Org. Synth. 2003, Vol. 80, 207-218. In this method, primary amines are converted to monoalkylhydroxylamines by a three step process which involves first, cyanomethylation of a primary amine, then the regioselective formation of a nitrone, followed by hydroxylaminolysis of this nitrone. The cyano group acts as a highly effective directing group for the regioselective formation of the nitrone, and so the desired product is obtained in high yields. However, the final stage of the process requires a hydroxylaminolysis step using high temperatures. Hydroxylamine has been shown to explode when heated under atmospheric pressure (Bretherick's Handbook of Reactive Chemical Hazards, 6^{th} Ed) and therefore, the operation of such a process, in particular on a large scale, can be hazardous.

The applicants have found an improved method for preparing hydroxylamines which can be used on a large scale, minimizes hazards and which allows access to enantiomerically pure substrates as indicated below.

According to the present invention, there is provided a process for preparing a salt of compound of formula (I) 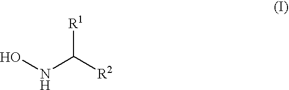
wherein R¹ and R² are independently selected from hydrogen or an organic group, said process comprising reacting a compound of formula (II) 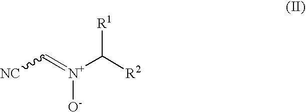
with water and an organic acid, in the absence of hydroxylamine.

Using this process, the compound of formula (I) obtained is in the form of a salt, which may be used directly in subsequent reactions, or it may, if required, be basified to access the free hydroxylamine.

In particular, the organic acid used in the process is p-toluenesulfonic acid (PTSA), but other acids such as oxalic acid or acetic acid may also be employed.

The reaction is suitably carried out in an organic solvent such as ethyl acetate, at moderate temperatures, for example from 20 to 60° C., in particular at about 40° C.

In particular this reaction will be useful in the preparation of compounds of formula (IA) 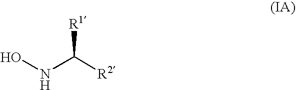
where R^{1′} and R^{2′} are equivalent to groups R¹ and R² as defined above, provided they are other than hydrogen and are different to each other. In this case, a compound of formula (IIA) will be used in the reaction 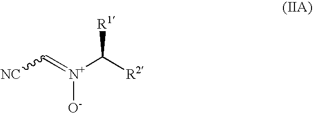
where R^{1′} and R^{2′} are as defined in relation to formula (IA).

Suitably the compound of formula (II) or (IIA) is obtained by reacting a compound of formula (III) or (IIIA) respectively 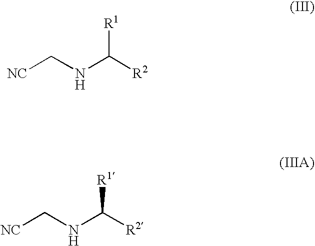
with an oxidizing agent, such as is m-chloroperbenzoic acid (MCPBA). Suitably the m-chloroperbenzoic acid is in the same organic solvent as is used in the reaction of compound of formula (II) or (IIA) and in particular this is ethyl acetate. Such a combination allows the reaction to proceed in the absence of environmentally less friendly solvents such as the halocarbons like dichloromethane, which has previously been used in this situation.

As indicated above, this reaction is regioselective, and therefore is particularly useful in the production of chiral compounds.

The reaction is suitably carried out at low temperatures for example from −10 to 20° C., in particular at about 5° C. The reaction is suitably worked up by washing with base, such as an alkali metal carbonate, bicarbonate or hydroxide, such as sodium bicarbonate, sodium carbonate or sodium hydroxide, and preferably sodium bicarbonate, added as an aqueous solution.

Suitably then the compound of formula (II) or (IIA) need not be isolated prior to this reaction, but can be reacted in situ, following washing with a basic solution and then brine, to produce the compound of formula (I) or (IA) respectively. The applicants have found that washing the product of the reaction between compound of formula (III) or (IIIA) and MCPBA with base removes the acid by-product, which minimizes product degradation and loss in yield. The aqueous waste from the washing regime can be tested for oxidants separately and treated accordingly.

Compounds of formula (III) or (IIIA) are suitably obtained by reacting a compound of formula (IV) or (IVA) respectively 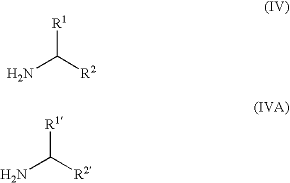
where R¹ and R² are as defined in relation to formula (I), and R^{1′} and R^{2′} are as defined in relation to formula (IA), with a compound of formula (V) 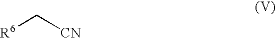
where R⁶ is a leaving group, such as halo and in particular bromo.

The reaction is suitably carried out in an organic solvent, in the presence of a base such as Hünig's base. If the organic solvent used here is the same as in the previous reactions, the entire sequence can be carried out simply, eliminating the need to remove solvents for example by evaporation, in order to effect a solvent swap. This is highly desirable, in particular where large-scale manufacture is undertaken.

Ethyl acetate has been found to be a particularly preferred solvent in this context, as it is environmentally more acceptable than some of the solvents such as the halocarbons like chloroform or dichloromethane, used in the previous methods for obtaining these compounds. Furthermore, in particular cases, it can be used throughout the process, avoiding the need for evaporation stages, such as rotary evaporation, which may be difficult to carry out, in particular on a large scale.

Suitable organic groups for R¹ and R² will be hydrocarbyl groups, which may optionally be substituted by functional groups, or which may contain heteroatoms such as oxygen, sulfur or nitrogen, provided the functional groups or the heteroatoms do not interfere with the reaction.

For instance, R¹ and R² may comprise alkyl, alkenyl, alkynyl, cycloalkyl, aryl, aralkyl or heterocyclic groups. Any of these may optionally be substituted by one or more functional groups. Examples of functional groups include halo, nitro, cyano, NR³R⁴, OR⁵, C(O)ₙR⁵, C(O)NR³R⁴, OC(O)NR³R⁴, NR⁵C(O)ₙR⁶, NR⁵C(O)NR³R⁴, N═CR⁵R⁶, S(O)ₘR⁵, S(O)ₘNR³R⁴ or —NR⁵S(O)ₙR⁶ where R³, R⁴, R⁵ and R⁶ are independently selected from hydrogen or optionally substituted hydrocarbyl, or R³ and R⁴ together with the atom to which they are attached, form an optionally substituted heterocyclyl ring as defined above which optionally contains further heteroatoms such as S(O)ₙ, oxygen and nitrogen, n is an integer of 1 or 2, m is 0 or an integer of 1-3.

Any cycloalkyl, aryl or heterocyclic groups may also be substituted by alkyl, alkenyl or alkynyl groups, which may themselves be optionally substituted by a functional group as described above.

Suitable optional substituents for hydrocarbyl groups R³, R⁴, R⁵ and R⁶ include halo, perhaloalkyl such as trifluoromethyl, mercapto, hydroxy, carboxy, alkoxy, heteroaryl, heteroaryloxy, alkenyloxy, alkynyloxy, alkoxyalkoxy, aryloxy (where the aryl group may be substituted by halo, nitro or hydroxy), cyano, nitro, amino, mono- or di-alkyl amino, alkylthio, alkylsulfinyl, alkylsulfonyl or oximino.

Where R³ and R⁴ together form a heterocyclic group, this may be optionally substituted by hydrocarbyl such as alkyl as well as those substituents listed above for hydrocarbyl groups R³, R⁴, R⁵ and R⁶.

As used herein, the expression “alkyl” includes groups having up to 10, preferably up to 6 carbon atoms, which may be both straight-chain and branched-chain alkyl groups such as propyl, isopropyl and tert-butyl. Similarly the terms “alkenyl” and “alkynyl” include unsaturated groups having from 2-10 and preferably from 2-6 carbon atoms, which may also be straight or branched. The term “cycloalkyl” includes C₃₋₈cycloalkyl groups such as cyclopropyl, cyclobutyl, cyclopentyl, cyclohexyl and cycloheptyl.

An analogous convention applies to other generic terms, for example “alkoxy” includes alkyl groups as defined above which are linked by way of an oxygen and so includes methoxy, ethoxy, propoxy, etc.

The term “aryl” refers to aromatic hydrocarbon rings such as phenyl or naphthyl. The terms “heterocyclic” or “heterocyclyl” include ring structures that may be mono- or bicyclic and contain from 3 to 15 atoms, at least one of which, and suitably from 1 to 4 of which, is a heteroatom such as oxygen, sulfur or nitrogen. Rings may be aromatic, non-aromatic or partially aromatic in the sense that one ring of a fused ring system may be aromatic and the other non-aromatic. Particular examples of such ring systems include furyl, benzofuranyl, tetrahydrofuryl, chromanyl, thienyl, benzothienyl, pyridyl, piperidinyl, quinolyl, 1,2,3,4-tetrahydroquinolinyl, isoquinolyl, 1,2,3,4-tetrahydroisoquinolinyl, pyrazinyl, piperazinyl, pyrimidinyl, pyridazinyl, quinoxalinyl, quinazolinyl, cinnolinyl, pyrrolyl, pyrrolidinyl, indolyl, indolinyl, imidazolyl, benzimidazolyl, pyrazolyl, indazolyl, oxazolyl, benzoxazolyl, isoxazolyl, thiazolyl, benzothiazolyl, isothiazolyl, morpholinyl, 4H-1,4-benzoxazinyl, 4H-1,4-benzothiazinyl, 1,2,3-triazolyl, 1,2,4-triazolyl, oxadiazolyl, furazanyl, thiadiazolyl, tetrazolyl, dibenzofuranyl, dibenzothienyl oxiranyl, oxetanyl, azetidinyl, tetrahydropyranyl, oxepanyl, oxazepanyl, tetrahydro-1,4-thiazinyl, 1,1-dioxotetrahydro-1,4-thiazinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

Where rings include nitrogen atoms, these may carry a hydrogen atom or a substituent group such as a C₁₋₆ alkyl group if required to fulfill the bonding requirements of nitrogen, or they may be linked to the rest of the structure by way of the nitrogen atom. A nitrogen atom within a heterocyclyl group may be oxidized to give the corresponding N-oxide.

The term “halo” or “halogen” includes fluorine, chlorine, bromine and iodine.

Suitably R¹ and R² are unsubstituted hydrocarbyl or heterocyclic groups.

In particular, one of R¹ or R² is an alkyl group, for example a C₁₋₃ alkyl group such as methyl, and the other is an aryl group such as phenyl or an aromatic heterocyclic group such as pyridyl.

Compounds obtained in accordance with the invention may have a wide range of applications. For example, chiral hydroxylamines have been used to prepare β-amino acids that can lead to modified peptides (H. S. Lee et al., J. Org. Chem. 2003, Vol. 68, No. 4, 1575-1578), as well as in the production of useful glycan derivatives (WO98/15566). They may also be used in the preparation of certain metalloproteinase inhibitors, for example, as described in a co-pending application of the applicants of even date to the present application.

The invention will now be particularly described by way of example.

### EXAMPLE 1

### Preparation of (S)-N-(1-Phenylethyl)hydroxylamine

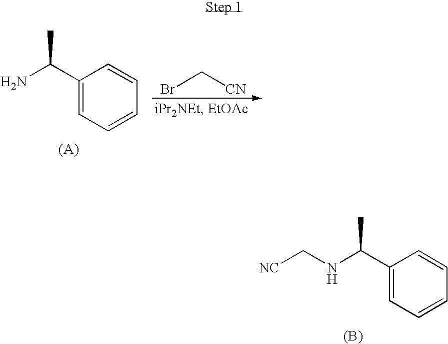

(S)-(−)-1-Phenylethylamine (Compound A) (4.70 g) was monoalkylated with bromoacetonitrile (5.12 g), in the presence of Hünig's base (7.6 mL) in ethyl acetate (27.5 mL) at 40° C. After 3 hours, water (7.5 mL) was added to dissolve the precipitated Hünig's base hydrobromide salt. The aqueous layer was removed and the organic layer containing Compound B was cooled to −2° C.

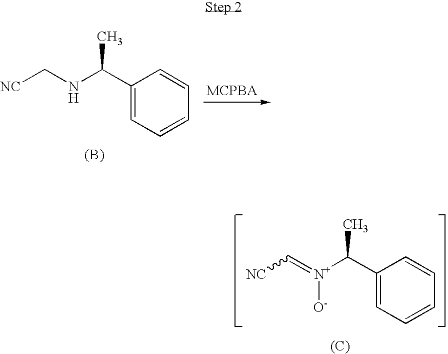

m-Chloroperbenzoic acid (MCPBA) (14.72 g) in ethyl acetate (30 mL) was added slowly to the organic phase from step 1 containing Compound B, so as to keep the reaction temperature below 5° C. The reaction mixture was washed sequentially with sodium bicarbonate (3×25 mL) and brine (25 mL) leaving a solution of Compound C in ethyl acetate.

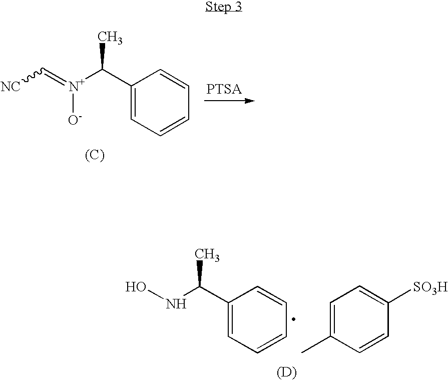

p-Toluenesulfonic acid monohydrate (PTSA) (7.38 g) was added to the organic phase from step 2 containing Compound C and the batch temperature heated at 40° C. for three hours. Compound D was then allowed to crystallize as the tosylate salt. The batch temperature is cooled to 0° C. and held for 1 hour. The product (Compound D) was collected by filtration and displacement washed with ethyl acetate, prior to drying in vacuo at 40° C. to a constant weight (8.56 g, 71% over 3 steps).

1. A process for preparing a salt of compound of formula (I) 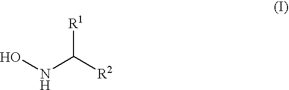
wherein R¹ and R² are independently selected from an organic group other than hydrogen, said process comprising reacting a compound of formula (II) 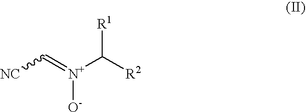
with water and an organic acid, in the absence of hydroxylamine.

2. A process according to claim 1 wherein the organic acid is p-toluenesulfonic acid.

3. A process according to claim 1 wherein the compound of formula (II) is a compound of formula (IIA) 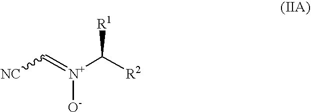
so that the product of formula (I) is a compound of formula (IA) 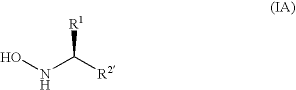
where R¹ and R² are as defined in claim 1 but are different.

4. A process according to claim 1 wherein the compound of formula (II) is obtained by reacting a compound of formula (III) 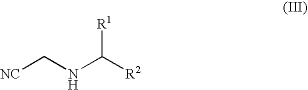
with an oxidizing agent.

5. A method according to claim 4 wherein the oxidizing agent is m-chloroperbenzoic acid.

6. A method according to claim 5 wherein the m-chloroperbenzoic acid is in ethyl acetate.

7. A method according to claim 1, wherein the compound of formula (III) is obtained by reacting a compound of formula (IV) 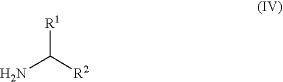
where R¹ and R² are as defined in relation to formula (I), with a compound of formula (V) 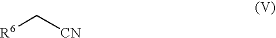
where R⁶ is a leaving group.

8. A method according to claim 1, wherein R¹ and R² are independently selected from unsubstituted hydrocarbyl or heterocyclic groups.

9. A method according to claim 1, wherein R¹ and R² are other than hydrogen and are different to each other.

10. A method according to claim 9 wherein one of R¹ or R² is an alkyl group, and the other is an aryl group or an aromatic heterocyclic group.
